(19) United States
(12) Patent Application Publication
(10) Pub. No.: US 2024/0321444 A1
(43) Pub. Date: Sep. 26, 2024

(54) MEDICAL IMAGING CONTROL APPARATUS, MEDICAL IMAGING SYSTEM, AND MEDICAL IMAGING CONTROL METHOD

(71) Applicant: Konica Minolta, Inc., Tokyo (JP)

(72) Inventors: Naoki HAYASHI, Higashimurayama-shi (JP); Satoshi HASEGAWA, Tokyo (JP); Akira HIROSHIGE, Kokubunji-shi (JP); Ichirou HAMAMOTO, Fuchu-shi (JP)

(21) Appl. No.: 18/603,812

(22) Filed: Mar. 13, 2024

(30) Foreign Application Priority Data
Mar. 23, 2023 (JP) 2023-046240

(51) Int. Cl. G16H 40/63; G16H 30/40

(52) U.S. Cl. CPC G16H 40/63; G16H 30/40

(57) ABSTRACT

A medical imaging control apparatus includes a hardware processor that causes a display to display a plurality of examinations of a same patient and different patients; specifies one examination from the plurality of examinations displayed on the display; causes the display to display a list of imaging order information corresponding to the specified one examination and imaging order information corresponding to another examination of the same patient as the one examination; and causes the display to display the imaging order information corresponding to the specified one examination so as to be distinguishable from the imaging order information corresponding to the other examination.

### REFERENCE TO RELATED APPLICATIONS

The entire disclosure of Japanese Patent Application No. 2023-046240, filed on Mar. 23, 2023, including description, claims, drawings and abstract is incorporated herein by reference in its entirety.

### BACKGROUND OF THE INVENTION

### Technical Field

The present invention relates to a medical imaging control apparatus, a medical imaging system, and a medical imaging control method.

### Description of Related Art

In X-ray imaging including general imaging, a person receives an examination order from a radiology information system (RIS) and performs imaging in accordance with the order. On the other hand, doctors who want to perform X-ray imaging are diverse, such as respiratory and cardiovascular physicians and orthopedic surgeons. Therefore, depending on the condition of the patient, examination orders from a plurality of departments may be placed on the same day.

However, while examination orders are issued by department, examinations are performed in units of examination orders on the console of the X-ray imaging system. For example, even if chest imaging is required for each of a respiratory organ and a circulatory organ for the same patient, there are two examination orders, and imaging is required twice.

On the other hand, a same-patient, multiple-examination display function is provided as operability (a function) that enables imaging to be carried out collectively for examinations of the same patient. The same-patient, multiple-examination display function is a function for shortening the imaging time (reducing the burden) for the target patient by collectively displaying the examinations of the same patient on an imaging screen, changing the imaging order across examinations, or duplicating a captured image for the same area conditions.

As the same-patient, multiple-examination display function, JP 4516464B describes collective displaying and switching the order after displaying. Also, JP 6397178B describes a state in which a plurality of orders are collectively displayed, and describes switching of the order of the display. Furthermore, J P 2021-118837A indicates that a plurality of orders are collectively displayed.

### SUMMARY OF THE INVENTION

An advantage of the same-patient, multiple-examination display function is that imaging orders belonging to a plurality of examinations for the same patient are collectively displayed in a list. With the same-patient, multiple-examination display function, a plurality of patients and a plurality of examinations are displayed in an examination list. When one examination is selected from among the plurality of examinations, the screen transitions to an imaging screen. On the imaging screen, the plurality of imaging orders for the same patient are displayed in a list.

However, since a plurality of examination orders are collectively displayed, a person may not know which examination has been selected on the examination list screen. The imaging order may not be identified because, for example, an imaging order for the same area is included in different examinations. It is likely to be difficult to understand which imaging order should have been selected first. None of JP 4516464B, JP 6397178B, and JP 2021-118837A discusses displaying an imaging screen in consideration of which examination order should be selected first when transitioning from the examination list to the imaging screen.

The present invention has been made in order to solve such a problem, and an object of the present invention is to provide a medical imaging control apparatus, a medical imaging system, and a medical imaging control method that can identify an appropriate imaging order among a plurality of imaging orders in a same-patient, multiple-examination display function for collectively displaying a plurality of examination orders for the same patient.

To achieve at least one of the abovementioned objects, according to an aspect of the present invention, a medical imaging control apparatus reflecting one aspect of the present invention includes: a hardware processor that causes a display to display a plurality of examinations of a same patient and different patients, specifies one examination from the plurality of examinations displayed on the display, causes the display to display a list of imaging order information corresponding to the specified one examination and imaging order information corresponding to another examination of the same patient as the one examination, and causes the display to display the imaging order information corresponding to the specified one examination so as to be distinguishable from the imaging order information corresponding to the other examination.

To achieve at least one of the abovementioned objects, according to an aspect of the present invention, a medical imaging system reflecting one aspect of the present invention is a medical imaging system comprising a medical imaging control apparatus to which a medical imaging apparatus is connected, the medical imaging control apparatus including: a hardware processor that causes a display to display a plurality of examinations of a same patient and different patients, specifies one examination from the plurality of examinations displayed on the display, causes the display to display a list of imaging order information corresponding to the specified one examination and imaging order information corresponding to another examination of the same patient as the one examination, and causes the display to display the imaging order information corresponding to the specified one examination so as to be distinguishable from the imaging order information corresponding to the other examination.

To achieve at least one of the abovementioned objects, according to an aspect of the present invention, a medical imaging control method reflecting one aspect of the present invention includes: by means of a hardware processor, displaying a plurality of examinations of a same patient and different patients on a display, specifying one examination from the plurality of examinations, displaying a list of imaging order information corresponding to the one examination and imaging order information corresponding to another examination of the same patient as the one examination on the display, and displaying the imaging order information corresponding to the one examination on the display so as to be distinguishable from the imaging order information corresponding to the other examination.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating a functional configuration of a medical imaging system according to the present embodiment;

FIG. 2 is an explanatory diagram illustrating an example of the display of an imaging control screen;

FIG. 3 is an explanatory diagram illustrating an example of the display of an examination list;

FIG. 4 is a block diagram illustrating an example of the configuration of the medical imaging control apparatus;

FIG. 5 is a flowchart illustrating an example of a medical imaging control method according to the present embodiment;

FIG. 6 is an explanatory diagram illustrating an example of a selection condition information settings screen; and

FIG. 7 is an explanatory diagram illustrating an example of switching of selection conditions in an examination list.

### DETAILED DESCRIPTION

Hereinafter, one or more embodiments of the present invention will be described with reference to the drawings. However, the scope of the invention is not limited to the disclosed embodiments.

### Example Overall Configuration of Medical Imaging System

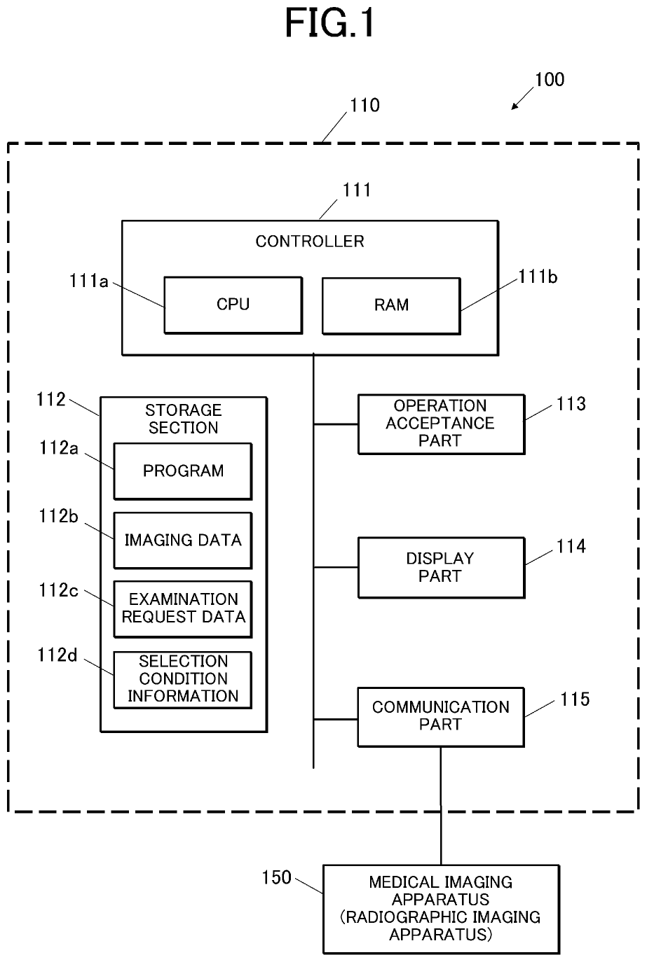

FIG. 1 is a block diagram illustrating a functional configuration of a medical imaging system according to the present embodiment. A medical imaging system 100 includes a medical imaging apparatus 150 and a medical imaging control apparatus 110. When the medical image is a radiographic (X-ray) image, the medical imaging apparatus is also referred to as a radiographic imaging apparatus. The medical imaging control apparatus is also referred to as a radiographic imaging control apparatus. Furthermore, the medical imaging system is also referred to as a radiographic imaging system.

The medical imaging control apparatus 110 includes a controller 111 (i.e., a hardware processor), a storage section 112 (i.e., a storage), an operation acceptance part 113 (i.e., an operation acceptor), a display part 114 (i.e., a display), and a communication part 115. The medical imaging control apparatus 110 may be, for example, a personal computer (PC).

The controller 111 controls the operation of each part of the medical imaging control apparatus 110. The controller 111 is a hardware processor including a central processing unit (CPU) 111a, random access memory (RAM) 111b, and the like. The CPU 111a performs arithmetic processing and performs various control operations. The RAM 111b provides a working memory space for the CPU 111a and stores temporary data.

The storage section 112 is nonvolatile memory and includes, for example, a hard disk drive (HDD) and/or flash memory. The storage section 112 stores a program 112a, imaging data 112b, examination request data 112c, selection condition information 112d, and the like.

The program 112a includes a program including processing related to a medical imaging control method according to the present embodiment. The program 112a is read and executed by the CPU 111a.

The imaging data 112b includes data of images captured by the medical imaging apparatus 150 and data of additional information associated with the captured image data. The additional information includes, for example, a reception number, patient ID, patient name, age, gender, date and time of imaging, and imaging conditions.

The examination request data 112c is data related to an examination request for medical imaging, such as radiographic imaging for example, acquired from a hospital information system (HIS), a radiology information system (RIS), or the like. The examination request data 112c includes information such as a reception number, patient ID, patient name, age, gender, examination registration date, requesting department, and imaging area, for example.

The selection condition information 112d is information used to specify one examination. The selection condition information will be described later.

The operation acceptance part 113 accepts an input operation from the outside by an examiner or the like who is a user of the medical imaging control apparatus 110, and outputs the input operation as an input signal to the controller 111. The operation acceptance part 113 includes, for example, some or all of a touch screen positioned to overlap the display part 114, a keyboard that accepts key operations, a pointing device such as a mouse, various switches and/or buttons, and the like.

The display part 114 is formed from a display such as a liquid crystal display (LCD), for example. The display part 114 displays an imaging control screen D which will be described later and the like under the control of the controller 111.

The communication part 115 controls communication and the transmission/reception of data with an external device or a peripheral device. For example, the communication part 115 connects to a local area network (LAN) by wired or wireless communication to perform communication. Apparatuses communicatively connected by the communication part 115 include the medical imaging apparatus 150.

### Example of Imaging Control Screen

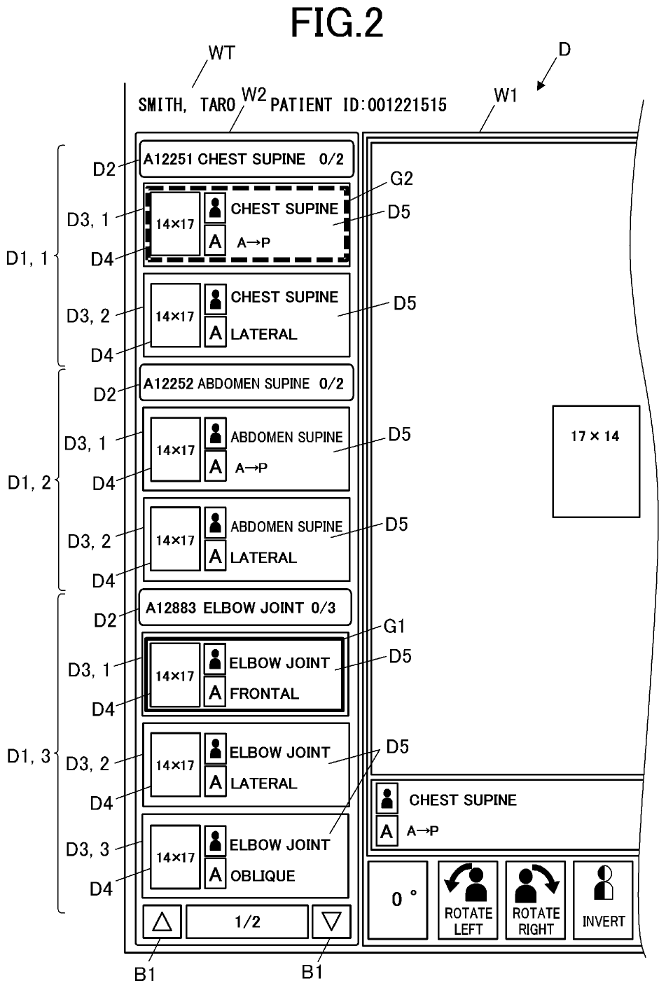

Next, the imaging control screen D to be displayed on the display part 114 of the medical imaging control apparatus 110 will be described. FIG. 2 is an explanatory diagram illustrating an example of the display of an imaging control screen. A title screen WT is displayed on the upper portion of the imaging control screen D. The title screen WT displays information specifying a patient to be imaged, such as a patient name and a patient ID. Furthermore, on the imaging control screen D, a captured image data preview screen W1 is displayed in the center, and a settings screen (not illustrated) is displayed on the right side thereof.

Furthermore, the imaging control screen D displays medical images set for the same patient on the left side of the preview screen W1. In the present example, a list screen W2 of examination information D1 related to the imaging of the radiographic images is displayed.

The examination information D1 displays a title field D2, in which a reception number, an imaging area, and the number of imagings are displayed, and imaging order information D3. The imaging order information D3 displays a thumbnail display field D4 and an information display field D5 indicating imaging information such as the imaging area and a summary of conditions. Note that the content of the title field D2 may be included in the imaging order information D3 and described together with the information display field D5. By writing the title field D2 in this way, a display format in which the title field D2 is not displayed may be used.

The examination information D1 is collected together by imaging area, for example. When one image is to be captured in one imaging area, one piece of imaging order information D3(1) is displayed for one piece of examination information D1. On the other hand, when a plurality of images are to be captured in one imaging area by changing the body position, a plurality of n pieces of imaging order information D3(1) to D3(n) are displayed for one piece of examination information D1. The examination information D1 is not limited to being collected together by imaging area, and may also be collected together in units of examinations.

The list screen W2 is switched between being displayed and not being displayed when there are too many pieces of examination information D1 to be displayed at once. The display of the examination information D1 is switched by operation of scroll buttons B1. Note that a slider bar may also be displayed beside the list screen W2. The display of the examination information D1 may also be switched by operation of the slider bar.

In the examination order information D3, an order that has already been imaged or has not been imaged yet can be identified by viewing the thumbnail display field D4, and an image can be copied on the imaging control screen D. As a result, an already captured image can be diverted to another examination, such as for imaging of the same area, and the number of imagings can be reduced.

Note that the display order of the examination order information D3 is according to the order of reception numbers in the present example. However, the display order may be any order such as the order of examination registration dates. Furthermore, the display order of the examination information D1 and the display order of the examination order information D3 can be changed after D1 and D3 are displayed. The display order of the examination information D1 and the display order of the examination order information D3 can also be changed to, for example, the order of imaging execution. Furthermore, although the examination order information D3 is a collection of the examination information D1 by imaging area, the examination order information D3 may also be listed without being collected by imaging area.

### Example of Examination List

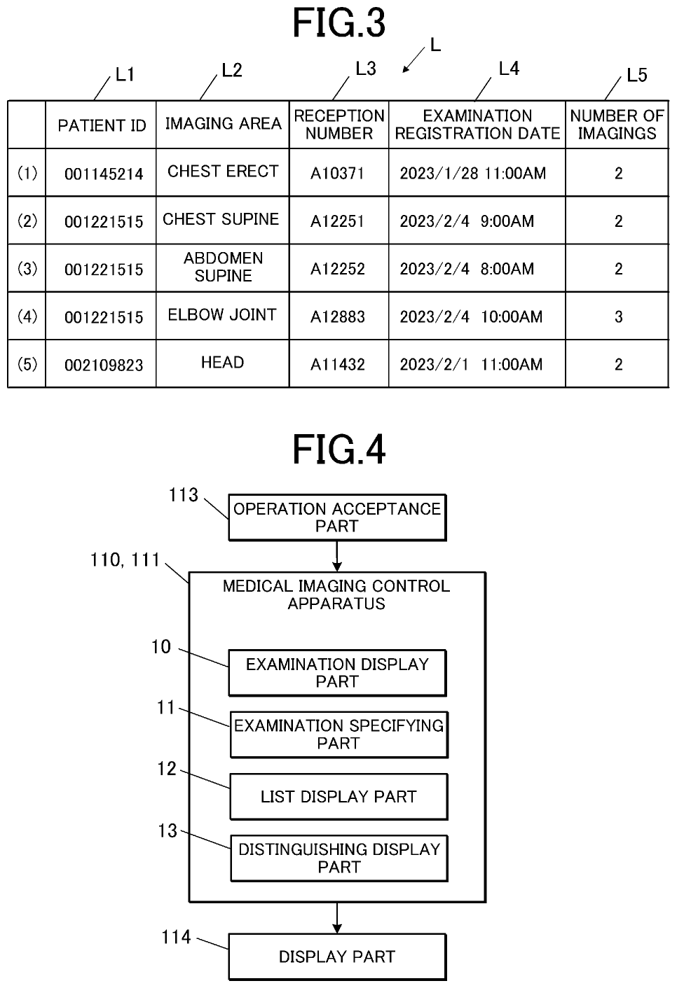

Next, the examination list L to be displayed on the display part 114 of the medical imaging control apparatus 110 will be described. FIG. 3 is an explanatory diagram illustrating an example of the display of an examination list. In the examination list L, patient ID information L1, imaging area information L2, reception number information L3, examination registration date information L4, and imaging number information L5 are displayed in an associated manner.

The examination list L specifies a patient ID, which is patient-specific identifying information, by the patient ID information L1. Further, the examination list L specifies the imaging area for each patient by the imaging area information L2. The examination list L specifies the reception number of the examination by the reception number information L3. Furthermore, the examination list L specifies the examination registration date by the examination registration date information L4. The examination list L specifies the number of imagings for each imaging by the imaging number information L5. When imaging is to be performed on different imaging areas of the same patient, the examination list L is created in which a reception number is assigned to each imaging area with the same patient ID.

### Functional Configuration Example of Medical Imaging Control Apparatus

As a function of enabling imaging for a plurality of examinations of the same patient at once, a same-patient, multiple-examination display function has been proposed. In the same-patient, multiple-examination display function, when one examination is selected on the examination list L in which a plurality of patients and a plurality of examinations are displayed, the screen transitions to the imaging control screen D. The imaging control screen D displays a list of all pieces of imaging order information for the patient corresponding to the selected examination.

The medical imaging control apparatus 110 specifies one examination from the plurality of examinations, and the one piece of imaging order information corresponding to the one examination is displayed so as to be distinguishable from the other imaging order information for the same patient.

FIG. 4 is a block diagram illustrating an example of the configuration of the medical imaging control apparatus. The medical imaging control apparatus 110 includes, as functional blocks, an examination display part 10, an examination specifying part 11, a list display part 12, and a distinguishing display part 13.

The examination display part 10 displays a plurality of examinations of the same patient and different patients as the examination list L illustrated in FIG. 3. The examination list L is an example of a first screen.

The examination specifying part 11 specifies one examination on the basis of predetermined selection condition information 112d, which is an example of supplementary information.

The list display part 12 specifies the patient by the patient ID corresponding to the selected examination. The selected examination is an examination selected by the operation accepted by the operation acceptance part 113, from among the plurality of patients and the plurality of examinations displayed in the examination list L. The list display part 12 acquires the imaging order information D3 corresponding to all examinations of patient identified by the patient ID corresponding to the selected examination, and displays a list of the imaging order information D3 on the imaging control screen D illustrated in FIG. 2. The imaging control screen D is an example of a second screen. The examination specifying part 11 specifies one examination on the basis of the selection condition information 112d, from among all examinations of the one patient specified by the patient ID corresponding to the selected examination. Thus, the imaging order information D3 corresponding to all the examinations of the same patient includes imaging order information D3(m) corresponding to the one examination specified by the examination specifying part 11. The imaging order information D3 corresponding to all examinations of the same patient includes imaging order information D3 corresponding to one examination and other examinations of the same patient.

The distinguishing display part 13 displays the imaging order information D3(m) corresponding to the one examination on the imaging control screen D so as to be distinguishable from the imaging order information D3 corresponding to other examinations. Displaying in a distinguishable manner refers to focus display. Focus display is, for example, to change the color of an area where the imaging order information D3(m) is displayed, or to provide a frame in which the imaging order information D3(m) is displayed. Other than focus display, methods of displaying in a distinguishable manner include displaying an arrow indicating the imaging order information D3(m), highlighting the reception number of the imaging order information D3(m), and the like. Further, methods of displaying the imaging order information D3(m) in a distinguishable manner also include a method such as displaying the imaging order information D3(m) with a predetermined word added thereto.

### Functional Configuration Example of Program

Next, the program 112a to be executed by the controller 111 will be described. The program 112a causes the controller 111 to execute a step of displaying a plurality of examinations of the same patient and different patients as the examination list L on the display part 114. The controller 111 functions as the examination display part 10 by causing the program 112a to execute the above step of displaying the examination list L on the display part 114.

The program 112a causes the controller 111 to execute a step of specifying one examination on the basis of the examination selected by the operation accepted by the operation acceptance part 113, from among the examinations displayed in the examination list L on the display part 114. The controller 111 functions as the examination specifying part 11 by causing the program 112a to execute the above step of specifying one examination.

Furthermore, the program 112a acquires the imaging order information D3 corresponding to all examinations of the patient to be subjected to the one examination. Next, the controller 111 is caused to execute a step in which the display part 114 displays a list on the imaging control screen D. The controller 111 functions as the list display part 12 by causing the program 112a to execute the above-described step of displaying the imaging order information D3(m) corresponding to the one examination and the imaging order information D3 corresponding to other examinations in the form of a list on the display part 113.

Furthermore, the program 112a causes the controller 111 to execute a step in which the display part 114 displays the imaging order information D3(m) corresponding to the one examination in a distinguishable manner on the imaging control screen D. The controller 111 functions as the distinguishing display part 13 by causing the program 112a to execute the above step of displaying the imaging order information D3(m) corresponding to the one examination in a distinguishable manner on the display part 114.

Some or all of these functions may be implemented by processing performed by a digital signal processor (DSP), instead of or in addition to the processing performed by the CPU 111a of the controller 111. Similarly, some or all of the functions may be implemented by processing performed by a dedicated hardware circuit instead of or in addition to processing performed by software.

Furthermore, the program is stored in the storage section 112 in advance, but is not limited thereto. For example, a recording medium in which these programs are recorded may be prepared, and the programs stored in this recording medium may be stored in the storage section 112. The recording medium in which these programs are recorded is, for example, an external recording medium such as a magnetic disk, an optical disc, or a solid-state drive (SSD). Alternatively, the program may be stored in a server connected to the medical imaging control apparatus 110 via a wired or wireless network, and the program may be transmitted to the storage section 112 via the network and stored in the storage section 112.

### Operation Examples of Medical Imaging Control Apparatus and Medical Imaging System According to Present Embodiment

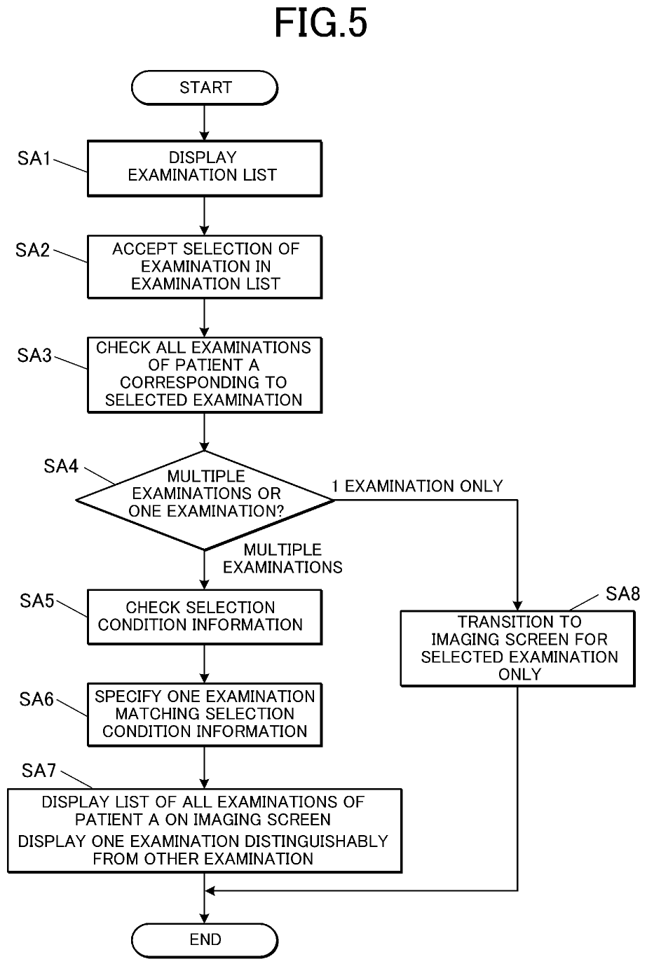

FIG. 5 is a flowchart illustrating an example of a medical imaging control method of the present embodiment. Next, as a medical imaging control method, an operation example of a program executed by the controller 111 of the medical imaging control apparatus 110 will be described with reference to the drawings.

In step SA1 of FIG. 5, the controller 111 causes the examination display part 10 to display the examination list L illustrated in FIG. 3 on the display part 114. In step SA2, the controller 111 accepts selection of an examination by the operation acceptance part 113 from a list of a plurality of patients and a plurality of examinations displayed in the examination list L. The user operates the operation acceptance part 113 to select a desired examination from the list of the plurality of patients and the plurality of examinations displayed in the examination list L.

In step SA3, the controller 111 confirms the presence or absence of a plurality of examinations corresponding to the same patient ID, on the basis of the patient ID corresponding to the examination that has been selected (selected examination). If the controller 111 determines in step SA4 that there are a plurality of examinations corresponding to the same patient ID, in step SA5, the examination specifying part 11 checks the selection condition information 112d.

In step SA6, the controller 111 specifies, by means of the examination specifying part 11, one examination matching predetermined selection condition information 112d from among all examinations of the same patient.

In step SA7, the controller 111 transitions to the imaging control screen D illustrated in FIG. 2, and the list display part 12 displays all the imaging order information D3 for the same patient on the list screen W2 of the imaging control screen D. Imaging order information D3 which cannot fit on the list screen W2 can be displayed by scrolling the list screen W2. Next, the controller 111 causes the distinguishing display part 13 to display the imaging order information D3(m) corresponding to the one examination in a distinguishable manner on the imaging control screen D. The controller 111 displays, for example, a distinguishing frame G1 around the imaging order information D3(m) corresponding to the one examination so as to be distinguishable from the other imaging order information D3.

In a case where a plurality of pieces of examination order information D1(1) to D3(n) exist with respect to one piece of examination information D1 on the imaging control screen D, the plurality of pieces of examination order information D3 are displayed in a vertical arrangement in the present example. Therefore, the imaging order information D3(m) corresponding to the one examination may be included in one piece of examination information D1 in which the plurality of pieces of examination order information D3(1) to D3(n) exist. In this case, the controller 111 displays the topmost examination order information D3(1) in a distinguishable manner.

If the controller 111 determines in step SA4 that there are not a plurality of examinations corresponding to the same patient ID, in step SA8, the controller 11 displays the imaging order information D3 corresponding to the selected one examination on the list screen W2 of the imaging control screen D.

### Example of Selection Condition Information

The selection condition information 112d is stored in the storage section 112. Examples of selection conditions defined by the selection condition information 112d include selected examination/examination ID/reception number/examination registration date/examination reservation date/imaging area/imaging procedure. As described above, the selected examination is an examination selected from the examinations displayed in the examination list L by an operation accepted by the operation acceptance part 113. The examination ID is identification information for each examination. Examination IDs are not assigned in the examination list L of FIG. 3. However, in a case where examination IDs are assigned, the selection condition information 112d may use the examination IDs as a selection condition.

The selection condition information 112d may be defined by a single selection condition or by combining a plurality of selection conditions. In a case where a plurality of selection conditions are combined, a preset order of priority may be added, and the imaging order information may be arranged according to the order of priority. The plurality of selection conditions may also be specified to be in ascending/descending order.

In a case where the selection condition is the imaging area, an order of priority may be set in advance for each area type, and the imaging order information may be arranged in order. For example, if the priority order is chest→abdomen→joint, the imaging order information is arranged in this order.

Furthermore, in a case where the selection condition is the imaging procedure (technique), imaging procedures may be specified according to a preset order of priority. For example, when an imaging system capable of serial imaging (dynamic imaging) or long-length imaging includes imaging conditions for serial imaging or long-length imaging, other selection conditions are not applied. The imaging system can also specify examination orders under this imaging condition as one examination and preferentially display the one examination in a distinguishable manner. Imaging procedures include techniques such as erect/supine/panel/long length/dynamic/contrast, and also directions such as frontal/lateral/P→A/A→P.

Also, an imaging execution status such as already-imaged/not-yet-imaged may be added to the selection conditions. Thus, setting conditions such as only not-yet-imaged, only already-imaged, both not-yet-imaged and already-imaged, not-yet-imaged with priority, and already-imaged with priority can be used as filtering conditions.

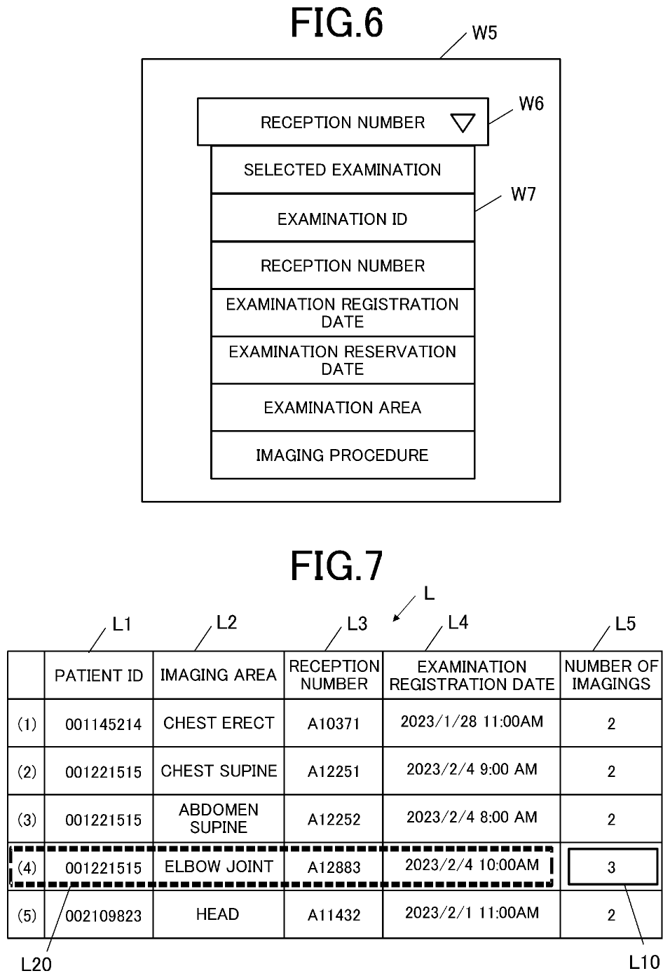

FIG. 6 is an explanatory diagram illustrating an example of a selection condition information settings screen. A selection condition information 112d settings screen W5 includes a settings display part W6 in which a selected selection condition is displayed, and a selectable selection condition list W7 is displayed as what is called a pull-down menu.

In this example, it is assumed that the examination selected in the examination list L is the examination (4) in the examination list L in FIG. 3. Furthermore, it is assumed that the selected examination is selected according to the selection condition information 112d. In this case, on the imaging control screen D, the imaging order information D3(1) of the examination information D1(3) in which the examination area is the elbow joint is displayed in a distinguishable manner by, for example, the distinguishing frame G1 as illustrated in FIG. 2.

When a reception number is selected according to the selection condition information 112d, the imaging order information D3(1) of the examination information D1(1) in which the examination area with the earliest reception number is the chest supine position is displayed in a distinguishable manner on the imaging control screen D illustrated in FIG. 2. The same applies to a case where the examination area is selected according to the selection condition information 112d in which the order of priority is chest→abdomen→joint, for example.

When an examination registration date is selected according to the selection condition information 112d, the imaging order information D3(1) of the examination information D1(2) in which the examination area with the earliest examination registration date (time) is the abdomen supine position is displayed in a distinguishable manner on the imaging control screen D illustrated in FIG. 2.

The examination selected in the examination list L (selected examination) and the one examination specified according to the selection condition information 112d (referred to as the specified examination) may be the same. For example, suppose a case where the examination selected in the examination list L is the examination (4) in the examination list L in FIG. 3 and the selected examination is selected according to the selection condition information 112d. In this case, the imaging order information D3(1) of the examination information D1(3) in which the examination area is the elbow is the imaging order information corresponding to the selected examination and corresponding to the specified examination matching the selection condition information 112d. Accordingly, when the selected examination and the specified examination matching the selection condition information 112d are the same, the controller 111 displays only the imaging order information D3(m) corresponding to the selected examination and the specified examination in a distinguishable manner.

On the other hand, the examination selected in the examination list L (selected examination) and the one examination specified according to the selection condition information 112d (specified examination) may be different. For example, suppose a case where the examination selected in the examination list L is the examination (4) in the examination list L in FIG. 3 and the reception number is selected according to the selection condition information 112d. In this case, the selected examination and the specified examination are different. If the selected examination and the specified examination are different, the controller 111 displays the imaging order information D3(m) corresponding to the specified examination and the imaging order information D3(k) corresponding to the selected examination so as to be distinguishable from each other. The controller 111 changes, for example, the color of the distinguishing frame G1 of the imaging order information G3(k) corresponding to the selected examination and the color of the distinguishing frame G2 of the imaging order information D3(m) corresponding to the specified examination. Furthermore, the line type of the distinguishing frame G1 and the line type of the distinguishing frame G2 may be changed.

It is assumed that the selected examination selected in the examination list L is the examination (4) in the examination list L in FIG. 3. In this case, the imaging order information D3(1) of the examination information D1(3) in which the examination area is the elbow joint is displayed in a distinguishable manner by the distinguishing frame G1 on the imaging control screen D.

Furthermore, it is assumed that the specified examination selected according to the selection condition information 112d is the examination (2) in the examination list L in FIG. 3. In this case, the imaging order information D3(1) of the examination information D1(1) in which the examination area is the chest supine position is displayed in a distinguishable manner by the distinguishing frame G2 on the imaging control screen D.

The controller 111 changes, for example, the color of the distinguishing frame G1 of the imaging order information corresponding to the selected examination and the color of the distinguishing frame G2 of the imaging order information corresponding to the specified examination. Further, the line type of the identification frame G1 and the line type of the identification frame G2 may be changed.

Furthermore, the fact that the imaging order information corresponding to the selected examination has been selected by a user operation may be displayed in a distinguishable manner. Also, the fact that the imaging order information corresponding to the specified examination has been selected on the basis of the selection condition information may be displayed in a distinguishable manner. Furthermore, the imaging order information corresponding to the selected examination need not be displayed in a distinguishable manner.

The selection condition of the selection condition information 112d may be changeable and retainable. The selection condition can be changed and retained not only for each medical imaging control apparatus 110 but also for each facility in which the medical imaging control apparatus 110 is installed and for each user who uses the medical imaging control apparatus 110. Thus, even if the efficient imaging sequence changes depending on the imaging room in which the medical imaging control apparatus 110 is installed or depending on the technician who uses the medical imaging control apparatus 110, it is possible to cope with this.

Further, sorting of the imaging order information D3 on the list screen W2 of the imaging control screen D can be linked with the selection condition of the selection condition information 112d. For example, in a case where the one examination (specified examination) is the chest frontal position, the imaging order information D3 is arranged so that imagings of the same area by the same procedure are performed consecutively, thereby enabling imaging in which the amount of patient movement (change in body position) is reduced as much as possible.

Further, the selection condition of the selection condition information 112d may be displayed in the examination list L. This allows the user (technician) to easily grasp in advance what will be in a selected state at the time of selection.

Furthermore, selection conditions of the selection condition information 112d may be switched by a selection operation on the examination list L. FIG. 7 is an explanatory diagram illustrating an example of switching of selection conditions in an examination list. For example, when the user selects the selected examination according to the selection condition information, the user selects a desired examination with a selection frame L10 indicated by a solid line. Also, when the user selects a selection condition other than the selected examination according to the selection condition information, the user selects a desired examination with a selection frame L20 indicated by a dashed line. Thus, it is possible to use different selection methods depending on the selection area and the operability of the selection method, such as double-clicking and long-pressing.

Note that in some conventional same-patient, multiple-examination display functions, processing is performed to delete the examination information itself for an examination for which no imaging order has been placed at the end of the examination. However, in the same-patient, multiple-examination display function, a plurality of examinations for the same patient are mixed together. Due to this mixing, it is difficult to recognize whether an imaging order remains for a specific examination, and there is a possibility that a non-executed examination order may be overlooked.

Therefore, the end of an examination may trigger the medical imaging system 100 to check whether there is an examination for which there are 0 pieces of imaging order information D3 among the plurality of pieces of examination information D1. If such an examination exists, the medical imaging system 100 may display a warning message. Alternatively, if such an examination exists, the medical imaging system 100 may interrupt the examination termination processing for deleting examination information. This keeps examination information with a remaining imaging order from being erroneously deleted. The medical imaging system 100 may allow for setting whether or not to execute processing for confirming whether or not there is an examination for which there are 0 pieces of imaging order information D3. Further, such a function may also be available only when the same-patient, multiple-examination display function is enabled.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-046240 filed on Mar. 23, 2023 is incorporated herein by reference in its entirety.

What is claimed is:

1. A medical imaging control apparatus comprising a hardware processor that:
causes a display to display a plurality of examinations of a same patient and different patients;
specifies one examination from the plurality of examinations displayed on the display;
causes the display to display a list of imaging order information corresponding to the specified one examination and imaging order information corresponding to another examination of the same patient as the one examination; and
causes the display to display the imaging order information corresponding to the specified one examination so as to be distinguishable from the imaging order information corresponding to the other examination.

2. The medical imaging control apparatus according to claim 1, wherein the hardware processor causes the display to display the imaging order information corresponding to the one examination in a manner different from the imaging order information corresponding to the other examination.

3. The medical imaging control apparatus according to claim 1, wherein:
the hardware processor causes the plurality of examinations to be displayed on a first screen;
the hardware processor causes the imaging order information corresponding to the one examination and the imaging order information corresponding to the other examination to be displayed in list form on a second screen; and
the hardware processor,
in response to a transition from the first screen to the second screen, causes the display to display at least one piece of imaging order information corresponding to the one examination so as to be distinguishable as being the imaging order information corresponding to the specified one examination.

4. The medical imaging control apparatus according to claim 1, further comprising a storage storing supplementary information pertaining to the plurality of examinations.

5. The medical imaging control apparatus according to claim 4, wherein the supplementary information is at least one of the following: patient-specifying information, an imaging area, examination identification information, an examination reception number, an examination registration date, an examination reservation date, and an imaging procedure.

6. The medical imaging control apparatus according to claim 4, wherein the hardware processor causes the display to display at least one piece of imaging order information corresponding to the one examination so as to be distinguishable as being the imaging order information corresponding to the specified one examination, on the basis of the supplementary information.

7. The medical imaging control apparatus according to claim 1, further comprising an operation acceptor that accepts an operation of selecting the one examination from the plurality of examinations,
wherein the hardware processor specifies the one examination on the basis of the operation of the operation acceptor.

8. The medical imaging control apparatus according to claim 4, wherein the hardware processor specifies the one examination from the plurality of examinations on the basis of the supplementary information.

9. A medical imaging system comprising a medical imaging control apparatus to which a medical imaging apparatus is connected,
the medical imaging control apparatus comprising a hardware processor that:
causes a display to display a plurality of examinations of a same patient and different patients;
specifies one examination from the plurality of examinations displayed on the display;
causes the display to display a list of imaging order information corresponding to the specified one examination and imaging order information corresponding to another examination of the same patient as the one examination; and
causes the display to display the imaging order information corresponding to the specified one examination so as to be distinguishable from the imaging order information corresponding to the other examination.

10. A medical imaging control method comprising, by means of a hardware processor,
displaying a plurality of examinations of a same patient and different patients on a display;
specifying one examination from the plurality of examinations;
displaying a list of imaging order information corresponding to the one examination and imaging order information corresponding to another examination of the same patient as the one examination on the display; and
displaying the imaging order information corresponding to the one examination on the display so as to be distinguishable from the imaging order information corresponding to the other examination.
